(19) United States
(12) Patent Application Publication
(10) Pub. No.: US 2026/0306244 A1
(43) Pub. Date: Oct. 8, 2026

(54) PROPHYLACTIC SYSTEM AND SUPPORT STRUCTURE FOR AN ANEURYSM

(71) Applicant: LIFE SEAL VASCULAR, INC., Costa Mesa, CA (US)

(72) Inventors: Michel Marinus Petrus Johannes REIJNEN, Arnhem (NL); William COLONE, Costa Mesa, CA (US); Meng TIA, Costa Mesa, CA (US); Paul REISS, Costa Mesa, CA (US)

(73) Assignee: LIFE SEAL VASCULAR, INC., Costa Mesa, CA (US)

(21) Appl. No.: 19/478,309

(22) PCT Filed: Apr. 26, 2024
(86) PCT No.: PCT/US2024/026486

§ 371 Date: Oct. 24, 2025

Related U.S. Application Data
(60) Provisional application No. 63/498,510, filed on Apr. 26, 2023.

(51) Int. Cl. A61F 2/07

(52) U.S. Cl. CPC A61F 2/07; A61F 2002/072; A61F 2002/077; A61F 2210/0014; A61F 2230/0069; A61F 2250/0037; A61F 2250/0098

(57) ABSTRACT

Systems and a support structure for treating an aneurysm are provided. A system includes a set of one or more stabilizing cylinders where each stabilizing cylinder includes a first sheet of expandable material, a second sheet of expandable material layered over the first sheet of expandable material, and a scaffold layer in between the first sheet of expandable material and the second sheet of expandable material. The combined layers including the first sheet of expandable material, the second sheet of expandable material, and the scaffold layer are arranged in a cylinder shape. Each stabilizing cylinder is configured to expand after being deployed to fill a space within an aneurysmal sac and is further configured to provide a space within the stabilizing cylinder for one or more structures.

This application claims the benefit of U.S. Provisional Patent Application No. 63/498,510, entitled as “PROPHYLACTIC SYSTEM AND SUPPORT STRUCTURE FOR AN ANEURYSM”, filed Apr. 26, 2023, which is incorporated by reference in its entirety.

### FIELD OF THE INVENTION

This disclosure relates to the field of treatment for aneurysms.

### BACKGROUND

Stent grafts are a common treatment for aortic aneurysms in general, whereby a stent graft is inserted into the aneurysms to facilitate the transport of blood through the aneurysm from the aortic artery to the iliac arteries, thereby depressurizing the aneurysm, and preventing aneurysm rupture. An endoleak is a leakage of blood into the space in the aneurysmal sac outside the stent graft, which may be referred to as the excluded aneurysm sac. The leakage of blood into the excluded aneurysm sac is classified into five types.

A type I endoleak occurs where blood leaks through at least one of the attachment sites at the aortic artery or iliac arteries. Type I endoleaks carry a high risk of aneurysm sac rupture because they tend to be high pressure. A type II endoleak occurs where blood flows into the excluded aneurysm sac through open collateral arteries. Type II endoleaks are lower pressure than type I and type III endoleaks. Type III endoleaks occur when blood leaks through the body of the stent graft into the excluded aneurysm sac. Type IV endoleaks occur due to porosity of the graft materials. Type V endoleaks occur where the aneurysmal sac grows without direct evidence of a leak. Although all types of endoleaks can become serious, type I and type III endoleaks generally require urgent medical attention. There is a need in the art for a solution that mitigates the risk of endoleaks and thus reduces a need for medical attention after installing a stent graft.

### SUMMARY

The disclosed subject matter comprises systems and a support structure for treating an aneurysm, adjunct to a conventional stent graft, to prevent endoleaks and promote aneurysm sac regression, thereby reducing reinterventions. An exemplary embodiment is a system for treating an aneurysm. The system includes a set of one or more stabilizing cylinders where each cylinder includes a first sheet of expandable material, a second sheet of expandable material layered over the first sheet of expandable material, and a scaffold layer in between the first sheet of expandable material and the second sheet of expandable material. The combined layers of the first sheet of expandable material, the second sheet of expandable material, and the scaffold are arranged in a cylinder shape. Each stabilizing cylinder is further configured to provide a space within the stabilizing cylinder for one or more structures. The set of one or more stabilizing cylinders may include two or more stabilizing cylinders. Lengths of the stabilizing cylinders, as measured along a flow of blood through the aneurysm, are tapered such that the lengths decrease from an inside stabilizing cylinder to an outside stabilizing cylinder. Tapering of the stabilizing cylinders may be configured to fit the stabilizing cylinders within a space in the aneurysm. The scaffold may include a material configured to self-expand after deployment in a blood vessel.

The scaffold may include nitinol. The system may further include a stent graft that is configured to be inserted inside the set of one or more stabilizing cylinders. The stent graft may be further configured to facilitate blood flow through the aneurysm. The scaffold layer of each stabilizing cylinder may be positioned such that the first sheet of expandable material and the second sheet of expandable material extend beyond the scaffold layer in a direction opposing a flow of blood in the aneurysm when the stabilizing cylinder is deployed. At least one stabilizing cylinder of the set of one or more stabilizing cylinders may include radiopaque markers.

Another general aspect is a stabilizing cylinder for treating an aneurysm. The stabilizing cylinder includes a first sheet of expandable material, a second sheet of expandable material layered over the first sheet of expandable material, and a scaffold layer between the first sheet of expandable material and the second sheet of expandable material. The combined layers of the stabilizing cylinder, which include the first sheet of expandable material, the second sheet of expandable material, and the scaffold, are arranged in a cylinder shape. The combined layers are configured to expand after being deployed to fill a space within an aneurysmal sac and are further configured to provide a space within the stabilizing cylinder for one or more structures. The stabilizing cylinder may be configured to be layered within the aneurysm with one or more additional stabilizing cylinders. Lengths of the stabilizing cylinders and one or more additional stabilizing cylinders, as measured along a flow of blood through the aneurysm, are tapered such that the lengths decrease from an inside stabilizing cylinder to an outside stabilizing cylinder.

Tapering of the stabilizing cylinders may be configured to fit the stabilizing cylinders within a space in the aneurysm. The scaffold layer may include material configured to self-expand after deployment and a blood vessel. The scaffold layer may include nitinol. The stabilizing cylinder may be configured to receive a stent graft positioned within the stabilizing cylinder to facilitate a flow of blood through the aneurysm. The scaffold layer may be arranged such that the first sheet of expandable material and the second sheet of expandable material extend beyond the scaffold layer in a direction opposing blood flow in the aneurysm when the stabilizing cylinder is deployed. The stabilizing cylinder may further include radiopaque markers. The first sheet of expandable material and the second sheet of expandable material may include a foam material.

An exemplary embodiment is a system for treating an aneurysm. The system includes a set of one or more stabilizing cylinders where each stabilizing cylinder includes a first sheet of expandable material, a second sheet of expandable material layered over the first sheet of expandable material, and a scaffold layer in between the first sheet of expandable material and the second sheet of expandable material. The combined layers of the first sheet of expandable material, the second sheet of expandable material, and the scaffold layer are arranged in a cylinder shape. Each stabilizing cylinder is configured to expand after being deployed to fill a space within an aneurysmal sac. Each stabilizing cylinder is further configured to provide a space within the stabilizing cylinder for one or more structures. The set of one or more stabilizing cylinders includes two or more stabilizing cylinders. Lengths of the stabilizing cylinders, as measured along a flow of blood through the aneurysm, are tapered such that the lengths decrease from an inside stabilizing cylinder to an outside stabilizing cylinder. Tapering of the stabilizing cylinders is configured to fit the stabilizing cylinders within a space in the aneurysm. The scaffold layer may include a nitinol material configured to self-expand subsequent to deployment in a blood vessel.

### BRIEF DESCRIPTION OF THE DRAWINGS

FIG. 1 is an illustration of the abdominal aortic artery, renal arteries, and bifurcation into the iliac arteries with representations of changes in the abdominal aortic artery during an aneurysm.

FIG. 2 is an illustration of a stent graft that has been implanted to treat an abdominal aortic aneurysm.

FIG. 3 is a cross-sectional view of a stabilizing cylinder showing a first expandable layer, a second expandable layer, and a scaffold between the expandable layers.

FIG. 4 is an illustration of a guide wire within an abdominal aortic aneurysm.

FIG. 5 is an illustration of a stabilizing cylinder that has been delivered to an abdominal aortic aneurysm.

FIG. 6 is an illustration of a second stabilizing cylinder that has been delivered to the abdominal aortic aneurysm.

FIG. 7 is an illustration of a third stabilizing cylinder that has been delivered to the abdominal aortic aneurysm.

FIG. 8 is an illustration of a stent graft that has been delivered to the abdominal aortic aneurysm after delivery of the three stabilizing cylinders.

FIG. 9 is a cross-sectional view of three stabilizing cylinders that have been delivered to an abdominal aortic aneurysm.

FIG. 10 is an image of a stabilizing cylinder within a transparent model of an abdominal aortic aneurysm.

FIGS. 11A and 11B are images of a stabilizing cylinder.

FIGS. 12A and 12B are images of a stabilizing cylinder from a side view next to a millimeter ruler.

### DETAILED DESCRIPTION

The disclosed subject matter is a stabilizing structure to prevent or diminish the severity of endoleaks subsequent to the deployment of a stent graft to an aneurysm. An exemplary embodiment of the disclosed subject matter comprises a stabilizing structure configured to surround a stent graft for an abdominal aortic aneurysm. In various embodiments, the stabilizing structure is configured to be positioned in the excluded aneurysm sac. There, the stabilizing structure may provide a stabilizing force for a stent graft and impede the flow of blood into the excluded aneurysm sac.

In an exemplary embodiment, a stabilizing cylinder is a layered structure comprising a first expandable layer, a second expandable layer, and a scaffold between the two expandable layers. The two expandable layers may comprise various expandable materials, such as foam. The scaffolding may include a self-expanding material such as nitinol. Treatment for an aneurysm may include various steps, including directing a guide wire to a position in an abdominal aortic aneurysm. The guidewire may guide a catheter carrying one or more components of a prophylactic treatment for an aneurysm.

In an exemplary embodiment, the components may include one or more stabilizing cylinders that surround a stent graft. Each of the one or more stabilizing cylinders may be delivered separately to the air aortic aneurysm. In various embodiments, an outermost stabilizing cylinder is delivered first. Once delivered, the stabilizing cylinder automatically expands to contact an inner wall of the abdominal aortic aneurysm. A second stabilizing cylinder is delivered into the hollow region within the expanded first stabilizing cylinder. Like the first stabilizing cylinder, the second stabilizing cylinder automatically expands once deployed within the abdominal aortic aneurysm. Any number of stabilizing cylinders may be delivered to the abdominal aortic aneurysm based on the size of the stabilizing cylinders and the shape of the abdominal aortic aneurysm. In an exemplary embodiment, three stabilizing cylinders are delivered to an abdominal aortic aneurysm. The third stabilizing cylinder is delivered within the hollow region of the expanded second stabilizing cylinder. Like the other two stabilizing cylinders, the third stabilizing cylinder automatically expands after delivery until it contacts the inner walls of the second stabilizing cylinder.

A stent graft may be delivered into the hollow region within the stabilizing cylinders. In the example given above, where three stabilizing cylinders are delivered to the abdominal aortic aneurysm, the stent graft is delivered into the hollow space within the three stabilizing cylinders and may be configured to expand until it makes contact with the third (innermost) stabilizing cylinder. Accordingly, the stabilizing cylinders provide stability to the stent to prevent movement and impede blood flow from the stent or feeder lumbar vessels into the abdominal aortic aneurysm.

In various embodiments, the two expandable layers comprise a porous spongelike material such as a foam. In an exemplary embodiment, the foam comprises biocompatible thermoset polycarbonate polyurethane. An exemplary embodiment of the two expandable layers comprises a resilient foam material that expands from a compressed state into an uncompressed state without permanent change to the structure of the foam material. The stabilizing cylinder may be delivered to a position of the abdominal aortic aneurysm while in a compressed state. After delivery, the stabilizing cylinder may expand into an uncompressed state or until the stabilizing cylinder contacts an inner barrier within the abdominal aortic aneurysm. The inner barrier may be an inner lumen of the abdominal aortic aneurysm, a thrombus, another stabilizing cylinder, another medical device, or the like.

In various embodiments, multiple stabilizing cylinders are used for a stent graft in an aneurysmal sac. The multiple stabilizing cylinders may be referred to herein as a support structure. The support structure may be delivered into the abdominal aortic aneurysm before delivering a stent graft. A hollow area in the center of the support structure, which comprises the space in the center of the three stabilizing cylinders, leaves space or an opening for a stent graft in the center of the aneurysm. In an exemplary embodiment, each of the layers of the stabilizing cylinders may be delivered in sequence where the outermost stabilizing cylinders are delivered to a position in the abdominal aortic aneurysm before stabilizing cylinders that are positioned inside. For example, in a stabilizing structure comprising three stabilizing cylinders, an outermost stabilizing cylinder may be delivered first, followed by a second middle stabilizing cylinder. The inner stabilizing cylinder is delivered last. The lengths of the stabilizing cylinders may vary depending on the size or shape of the aneurysm based on a patient scan.

In various embodiments, a catheter may deliver layers of the stabilizing cylinder in a compressed state. The multiple components of the stabilizing structure may be held in the compressed state by multiple means, including through compressive force from the inner walls of the catheter. In an exemplary embodiment, the components of the stabilizing structure may be pushed out of the catheter at the delivery location by a plunger.

In various embodiments, each of the stabilizing cylinders of the stabilizing structure has a different length as measured along an axis from the upper portion of the abdominal aortic artery to the bifurcation of the abdominal aortic artery into the iliac arteries. The varied lengths may be configured to fit the shape of the aortic aneurysm. For example, where the stabilizing structure has three stabilizing cylinders, the first (outer) stabilizing cylinder, which is positioned on the outside of the stabilizing structure, has the shortest length. The second (middle) stabilizing cylinder may have a longer length than the first stabilizing cylinder, and the third (inner) stabilizing cylinder may have the longest length of the three stabilizing cylinders. In various embodiments, the lengths of each of the one or more stabilizing cylinders may be tailored to fit the dimensions of the abdominal aortic aneurysm for each patient. In various embodiments, one or more stabilizing cylinders may include radiopaque markers that enhance visualization of the stabilizing cylinders under multiple forms of spectroscopy.

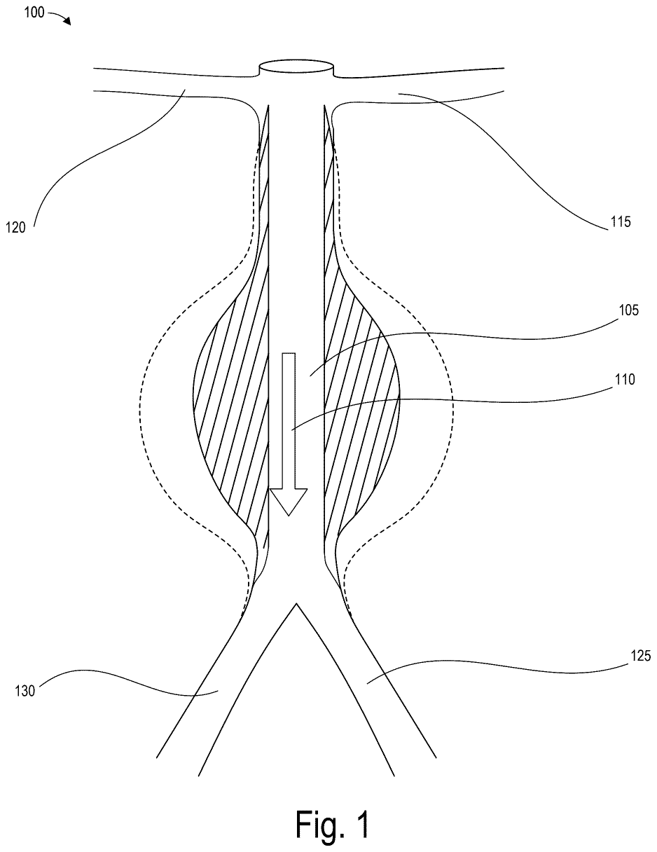

Referring to FIG. 1, FIG. 1 is an illustration 100 of the abdominal aortic artery, renal arteries, and bifurcation into the iliac arteries with representations of changes in the abdominal aortic artery during an aneurysm. In the perspective shown in the illustration 100, the aortic artery 105 pumps blood from the heart in a downward direction 110. The left renal artery 115 and right renal artery 120 branch from the aortic artery 105, as shown in the upper portion of the illustration 100. The lower portion of the illustration 100 shows the bifurcation of the aortic artery 105 into the left iliac artery 125 and the right iliac artery 130. Multiple collateral vessels have access to the aortic artery 105 between the renal and iliac arteries. The most common collateral vessels are the lumbar arteries and mesenteric arteries. The collateral vessels and arteries can profuse blood into the aneurysm sac via a Type II endoleak.

An abdominal aortic aneurism is an expansion of the aortic artery 105. It usually occurs in the portion of the aortic artery 105 between the renal arteries and the bifurcation of the left iliac artery 125 and right iliac artery 130. The expansion of the aortic artery can potentially result in a rupture, which is a life-threatening condition.

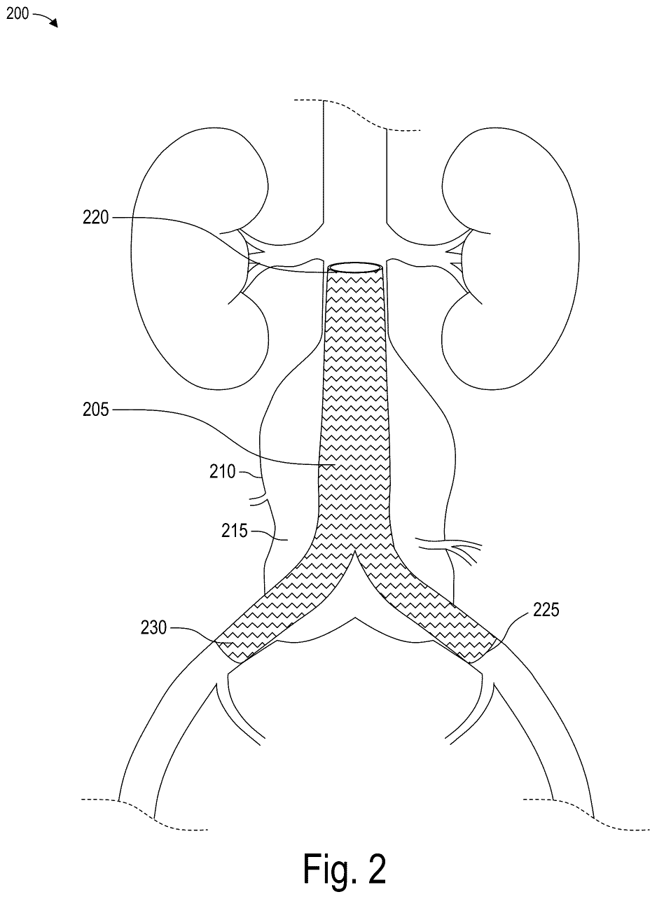

Referring to FIG. 2, FIG. 2 is an illustration 200 of a stent graft 205 implanted to treat an abdominal aortic aneurysm 210. The space of the abdominal aortic aneurysm 210 between the stent graft 205 and the inner wall of the abdominal aortic aneurysm 210 may be referred to as the excluded aneurysm sac 215. An endoleak, whereby blood fills the excluded aneurysm sac 215, may lead to the expansion of the abdominal aortic aneurysm and potentially lead to a life-threatening rupture.

There are multiple types of endoleaks depending on the path that blood takes to enter the excluded aneurysm sac 215. When blood enters the excluded aneurysm sac 215 through one of the attachment points of the stent graft to the blood vessels, it is considered a type I endoleak. For instance, blood may enter the excluded aneurysm sac 215 through the attachment point 220 of the stent graft 205 to the aortic artery, where blood enters the stent graft 205. Blood may also enter the excluded aneurysm sac 215 through the left iliac artery attachment point 225 or right iliac artery attachment point 230 to the stent graft 205.

When blood enters the excluded aneurysm sac 215 through one of the collateral vessels such as, but not limited to, the mesenteric artery or lumbar artery, it is considered a type II endoleak, which is not as severe as a type I endoleak, but could require medical attention nonetheless. When blood enters the excluded aneurysm sac 215 through a defect in the material of the stent graft 205, it is considered a type III endoleak. Type I and III endoleaks can result in high pressure inside the aneurysm that can quickly lead to a rupture of the abdominal aortic aneurysm.

When blood enters the excluded aneurysm sac 215 through pores in the material of the stent graft 205, it is considered a type IV endoleak. When blood enters the excluded aneurysm sac 215 through an unknown pathway, it is regarded as a type V endoleak. The support structure described herein can help stabilize type IV and type V endoleaks.

The disclosed stabilizing structure helps prevent movement (lateral and longitudinal) of the stent graft 205, potentially resulting in type I or type III endoleaks. Further, the disclosed stabilizing structure applies constant pressure to open spaces in the excluded aneurysm sac 215, potentially mitigating blood flow into the excluded aneurysm sac 215 for all types of endoleaks.

An artificial lumen, or lumen structure, facilitates blood flow through a blood vessel that may be positioned such as through the aneurysm shown in the illustration 200. There are many variations of lumen structures to which the disclosed subject matter may be applied. One type of lumen structure is a stent graft 205, which comprises a mesh tube (stent) surrounded by material (graft).

An example of a stent is a mesh that is configured to be delivered to a blood vessel in a compressed state. Once delivered, the stent may be expanded into a tube shape that approximates a blood vessel. Various stent materials comprise a metal material, such as nitinol, that expands into a predefined shape when exposed to blood after being delivered by a catheter. Other stent meshes, such as those made with stainless steel, are expanded manually via balloons or push wires.

The graft material may be any material that surrounds the stent mesh and impedes blood from traversing the walls of the graft material. The inside surface of the graft material creates the inner lumen of the lumen structure and encounters blood that flows through it. The outside surface of the graft structure contacts the disclosed stabilizing structure for the aneurysm. Various graft materials include but are not limited to polyester, polytetrafluoroethylene (PTFE or ePTFE), and polyethylene terephthalate (PET). The graft material is typically attached to the stent prior to delivery of the stent graft. Thus, the graft is compressed or folds with the stent during delivery and expands or unfolds as the stent expands within the blood vessel.

The stent graft 205 may be anchored within the blood vessel to prevent movement or migration of the stent graft 205. In particular, the attachment points of the stent graft 205, where blood enters and exits the stent graft 205, should be secure to the walls of the blood vessel to prevent leakage of the blood to the damaged portion of the blood vessel. Anchoring may be accomplished by various means, including but not limited to mechanical pressure from the expanded stent against the inner lumen of the blood vessel, barbs in the stent graft 205 that pierce the blood vessel, and another medical device that provides an anchor.

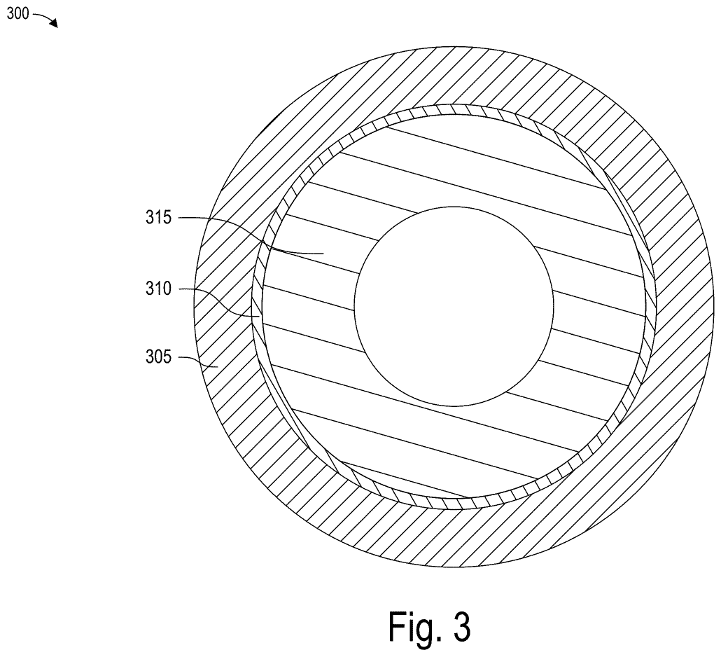

Referring to FIG. 3, FIG. 3 is a cross-sectional view of a stabilizing cylinder 300 showing a first expandable layer 305, a second expandable layer 315, and a scaffold 310 in between the expandable layers. The first expandable layer 305 and second expandable layer 315 may comprise a porous and expandable material such as a foam. In various embodiments, the foam comprises biocompatible thermoset polycarbonate polyurethane and other similar materials.

A porosity of the expandable material in the expandable layers may allow blood that leaks into the aneurysmal sac to enter the expandable layer, become trapped, and coagulate.

Accordingly, the aneurysmal sac does not grow because coagulated blood prevents more blood from entering the aneurysmal sac.

In various embodiments, the scaffold 310 comprises a wire mesh that is sandwiched between the first expandable layer 305 and the second expandable layer 315. In an exemplary embodiment, the wire mesh comprises a metal material, such as nitinol, that self-expands to a predefined shape when the temperature of the metal material meets a threshold. In various embodiments, the first expandable layer 305 and second expandable layer 315 extend a short distance beyond the scaffold 310 at one end of the stabilizing cylinder 300. In other embodiments, the wire mesh may be attached to the inner surface of an expandable foam layer. Accordingly, only a single expandable foam layer may be used to reduce the size of the delivered support structure.

In an exemplary embodiment, the stabilizing cylinder 300 is deployed in the abdominal aortic aneurysm such that an upper end of the stabilizing cylinder 300, at which blood enters the stabilizing structure, comprises the first expandable layer 305 and second expandable layer 315 that extends a distance beyond the scaffold 310. All three layers of the stabilizing cylinder 300 may be aligned at the lower end of the stabilizing cylinder 300, at which blood exits the stabilizing structure.

Space in the middle of the stabilizing cylinder may be hollow, allowing various structures (e.g., stent grafts and the like) to be placed inside. In an exemplary embodiment, a first stabilizing cylinder may be deployed in an abdominal aortic aneurysm, followed by a second stabilizing cylinder, which is deployed inside the first stabilizing cylinder. A third stabilizing cylinder may then be deployed inside the second stabilizing cylinder. A stent graft is then deployed inside the third stabilizing cylinder. The three stabilizing cylinders provide a continuous stabilizing force to maintain the stent graft in its original position within the abdominal aortic aneurysm. Further, the three stabilizing cylinders block the flow of blood into the excluded aneurysm sac 215 to either stop the blood or reduce the flow of blood, thus reducing the potential damage as a result of the endoleak.

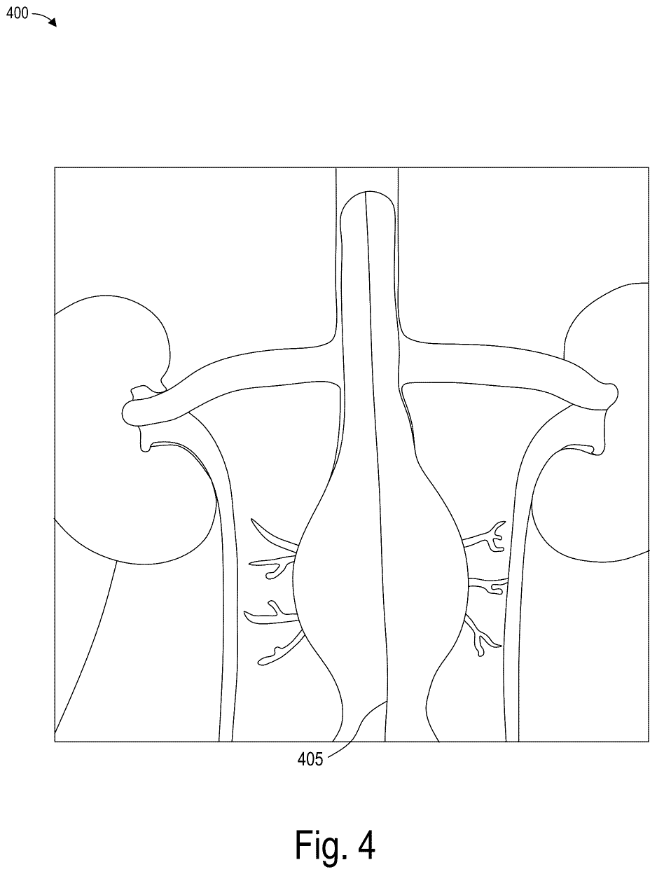

Referring to FIGS. 4-8, FIGS. 4-8 show a sequence of images that illustrate a deployment of the stabilizing structure and a stent graft in an abdominal aortic aneurysm. The stabilizing structure comprises a set of one or more stabilizing cylinders that are configured to expand until they meet a barrier, such as an inner lumen or an inner wall of another stabilizing cylinder. Each stabilizing cylinder has a hollow core that allows one or more structures to be inserted into the hollow core.

Referring to FIG. 4, FIG. 4 is an illustration 400 of a guide wire 405 deployed within an abdominal aortic aneurysm. In the example shown in the illustration 400, the guide wire 405 is deployed from a blood vessel that is downstream from the abdominal aortic aneurysm. The guide wire 405 is subsequently delivered to the desired location for deployment of the stabilizing structure and stent graft.

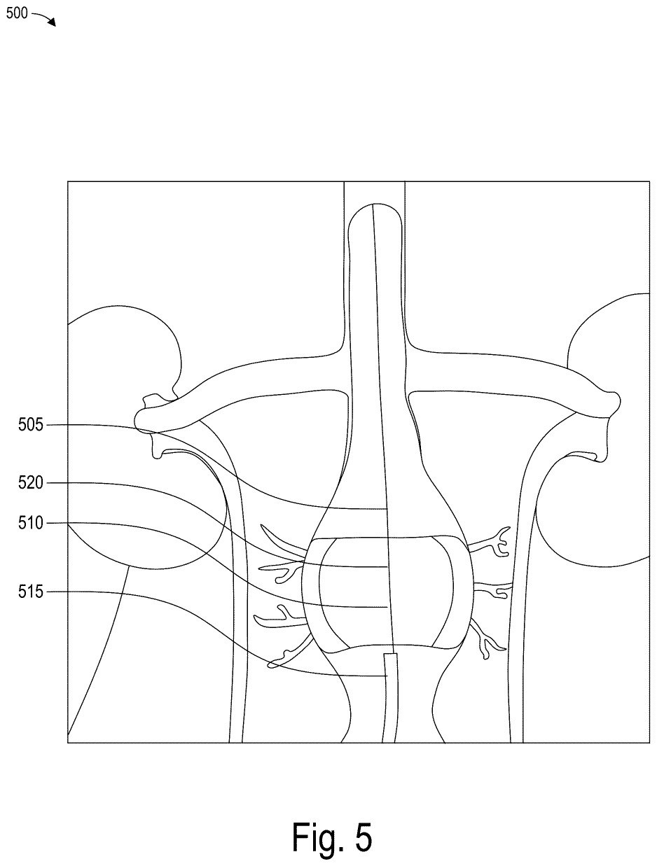

Referring to FIG. 5, FIG. 5 is an illustration 500 of a stabilizing cylinder that has been delivered to an abdominal aortic aneurysm. The stabilizing cylinder 510 may be delivered to the abdominal aortic aneurysm by a delivery catheter 515. In an exemplary embodiment, the stabilizing cylinder 510 is compressed inside the delivery catheter 515, whereby the delivery catheter 515 is positioned at a delivery location by the guide wire 505.

The stabilizing cylinder 510 may be pushed out of the delivery catheter 515 by various means, such as a plunger. Subsequent to delivery, the stabilizing cylinder 510 may expand from its compressed state to an expanded state. Accordingly, the stabilizing cylinder 510 may expand until an outer wall of the stabilizing cylinder makes contact with an inner lumen of the abdominal aortic aneurysm. In various embodiments, the stabilizing cylinder 510 will make contact with one or more blood clots inside the abdominal aortic aneurysm instead of contacting an inner lumen.

The length of the stabilizing cylinder 510 may be customized based on the dimensions of the abdominal aortic aneurysm for an individual patient. In various embodiments, the first stabilizing cylinder to be delivered to the abdominal aortic aneurysm will have the shortest length as it corresponds to the geometry of the abdominal aortic aneurysm. The stabilizing cylinder 510 may be a hollow space 520, which allows for various structures to be inserted within the stabilizing cylinder 510.

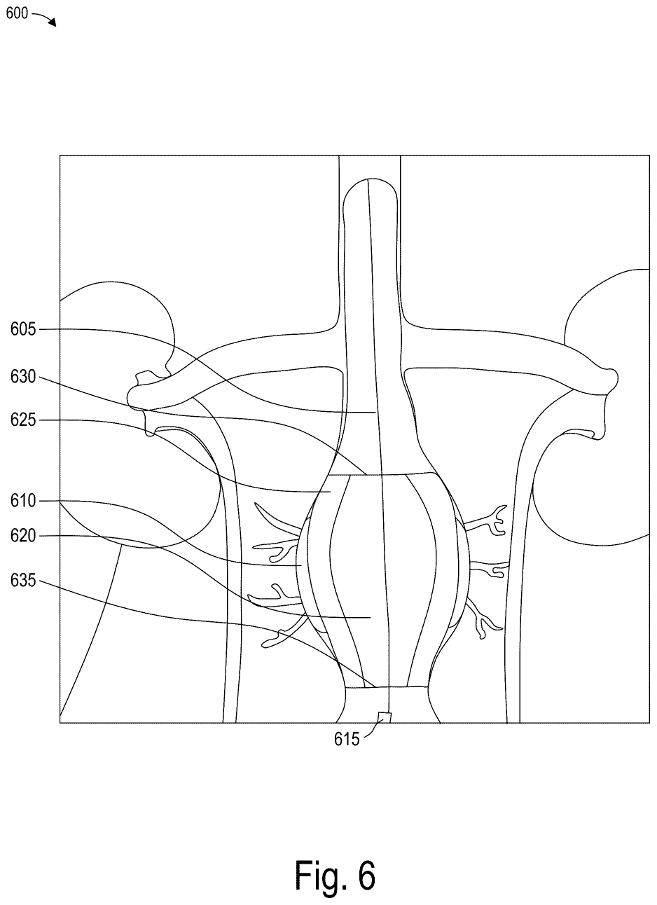

Referring to FIG. 6, FIG. 6 is an illustration 600 of a support structure subsequent to a second stabilizing cylinder 625 being delivered to the abdominal aortic aneurysm. The second stabilizing cylinder 625 may be delivered into the hollow space 520 of the first stabilizing cylinder 610. Like the first stabilizing cylinder 610, the second stabilizing cylinder 625 may be deployed by a delivery catheter 615 inside the abdominal aortic aneurysm. In various embodiments, the same delivery catheter 615 and guide wire 605 that were used to deploy the first stabilizing cylinder 610 may be used to deploy the second stabilizing cylinder 625.

In an exemplary embodiment, a stabilizing cylinder that is delivered to an abdominal aortic aneurysm may have a greater length than subsequently delivered stabilizing cylinders. As shown in the illustration 600, the second stabilizing cylinder 625 is longer in length as measured from an upper end 630 of the stabilizing cylinder to a lower end 635 of the stabilizing cylinder as compared to this first stabilizing cylinder 610. In various embodiments, each subsequently delivered stabilizing cylinder may be longer in length than the previously delivered stabilizing cylinders.

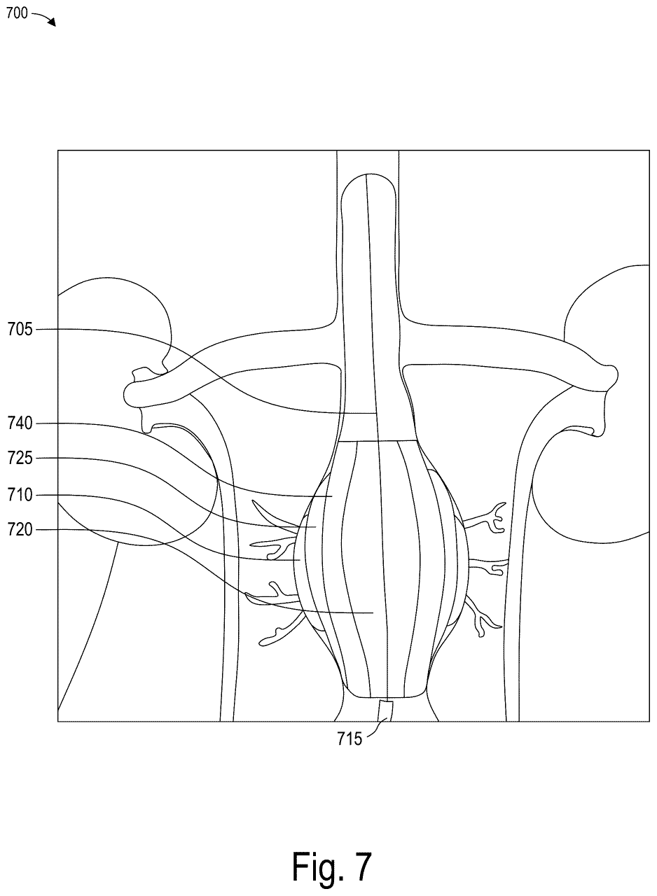

Referring to FIG. 7, FIG. 7 is an illustration 700 of a third stabilizing cylinder 740 that has been delivered to the abdominal aortic aneurysm. Like the second stabilizing cylinder 725, the third stabilizing cylinder may be delivered into the hollow space 620 of the second stabilizing cylinder 725. In the exemplary embodiment shown in FIG. 7, the third stabilizing cylinder 740 is longer in length than the second stabilizing cylinder 725, which in turn, is longer in length than the first stabilizing cylinder 710. As such, the support structure created by the first stabilizing cylinder 710, second stabilizing cylinder 725, and third stabilizing cylinder 740 more or less conform to the dimensions of the abdominal aortic aneurysm.

The third stabilizing cylinder, 740, may be delivered by a delivery catheter 715. In various embodiments, the same delivery catheter 715 that delivered the first stabilizing cylinder 710 and second stabilizing cylinder 725 may deliver the third stabilizing cylinder 740 to the abdominal aortic aneurysm. Likewise, the same guide wire 705 as was used with the previous stabilizing cylinders may be used to position the delivery catheter 715 to deliver the third stabilizing cylinder 740. The third stabilizing cylinder includes hollow space 720, which allows for one or more structures to be delivered to the abdominal aortic aneurysm.

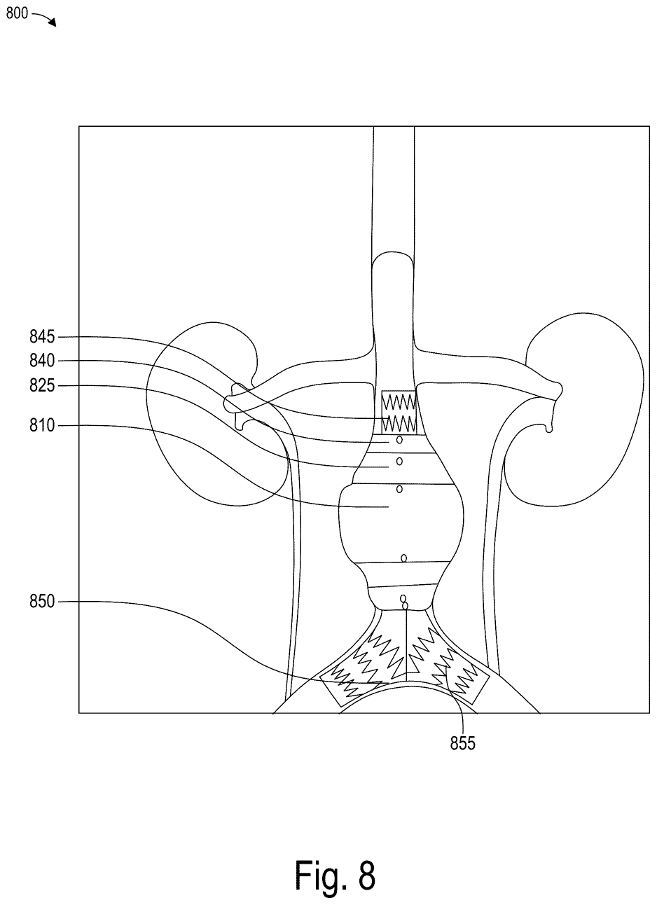

Referring to FIG. 8, FIG. 8 is an illustration 800 of a stent graft 845 that has been delivered to the abdominal aortic aneurysm subsequent to delivery of the three stabilizing cylinders. The stent graft 845 may be delivered into the hollow space 720 made by the third stabilizing cylinder 840. In an exemplary embodiment, the stent graft 845 may be delivered by the same delivery catheter 715 that delivered the first stabilizing cylinder 810, second stabilizing cylinder 825, and third stabilizing cylinder 840.

The stent graft 845 may comprise various configurations comprising various shapes and sizes. The disclosed stabilizing structure may be shaped to be configured to work with many types of stent grafts 845. The stent graft 845 in illustration 800 comprises a three-part structure, including a main portion that branches into a left bifurcated portion 855 and a right bifurcated portion 850. The stabilizing structure wraps around the three parts of the stent graft 845 and secures the stent graft 845 to a position within the aneurysm.

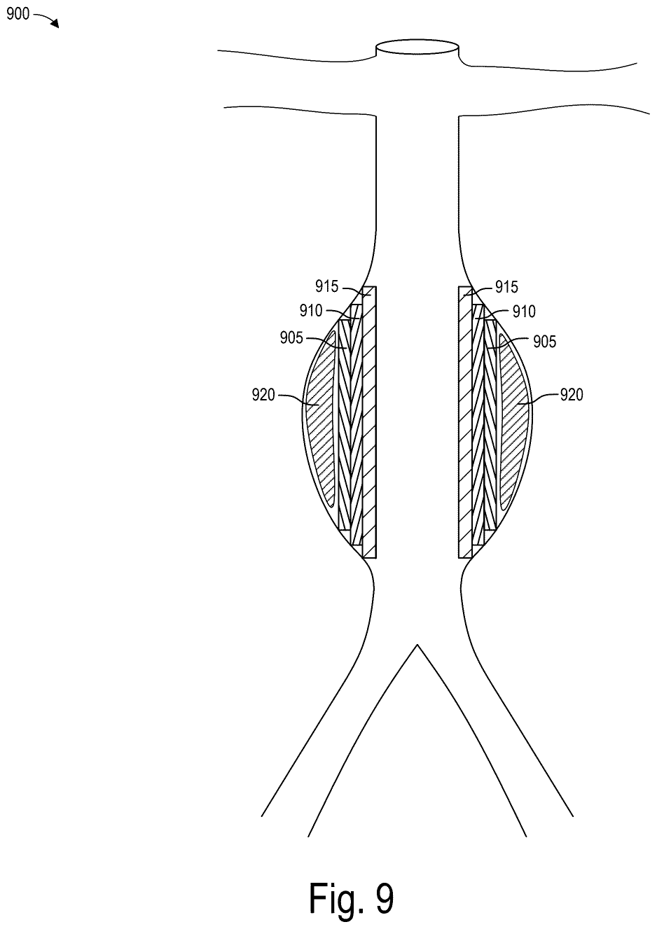

Referring to FIG. 9, FIG. 9 is a cross-sectional view 900 of three stabilizing cylinders that have been delivered to an abdominal aortic aneurysm. The stabilizing structure shown in the cross-sectional view 900 includes a first stabilizing cylinder 905, a second stabilizing cylinder 910, and a third stabilizing cylinder 915. When the stabilizing cylinders are delivered, they may expand until they make contact with an inner lumen, another stabilizing cylinder, or another object.

An abdominal aortic aneurysm will be unique for every patient. The shape of the abdominal aortic aneurysm and other factors may determine a unique geometry that will be filled by the stabilizing structure. In many cases, the patient may have one or more blood clots or thrombi within the abdominal aortic aneurysm. In the cross-sectional view 900 shown in FIG. 9, the abdominal aortic aneurysm includes a thrombus 920 around the outer wall of the abdominal aortic aneurysm. Accordingly, the first stabilizing cylinder 905 is in an expanded state whereby it expanded until it may contact with the thrombus 920.

The stabilizing cylinders are oriented such that the axis of each stabilizing cylinder is aligned with the flow of blood through the abdominal aortic aneurysm. Accordingly, the length of the stabilizing cylinders is customized to fit the dimensions of the abdominal aortic aneurysm, which includes the thrombus. As shown in the cross-sectional view 900, the length of the first stabilizing cylinder 905 is shorter than the length of the second stabilizing cylinder 910. Likewise, the length of the second stabilizing cylinder 910 is shorter than the length of the third stabilizing cylinder 915. The stabilizing structure is not limited to a set of 3 stabilizing cylinders. In various embodiments, the set of stabilizing cylinders may comprise any number of stabilizing cylinders. In the cross-sectional view 900, an additional stabilizing cylinder could be added to the stabilizing structure if the thrombus 920 were not present.

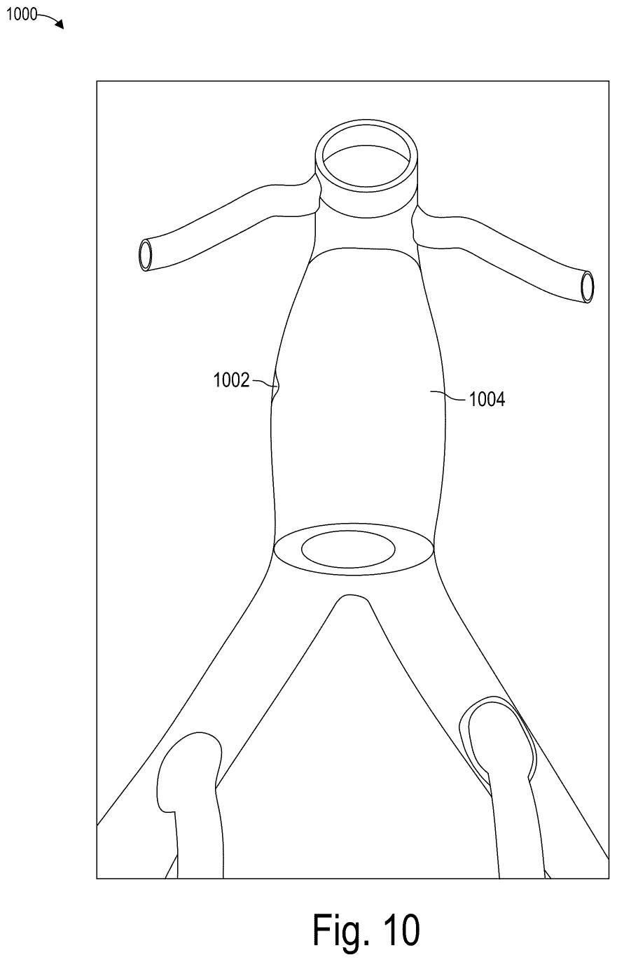

Referring to FIG. 10, FIG. 10 is an image 1000 of a prototype of a system for treating an aneurysm. In one embodiment, the aneurysm is an abdominal aortic aneurysm (AAA). The abdominal aortic aneurysm is an expanded area, as shown in 1002, in a lower portion of a main artery, which is known as the aorta. The aorta is the main blood vessel that supplies blood from the heart to the abdomen, pelvis, and legs.

The prototype of the system is made of glass material to illustrate the abdominal aortic aneurysm. As shown in FIG. 10, the system for treating the aneurysm includes a stabilizing cylinder 1004. The stabilizing cylinder 1004 includes three layers, as shown in FIG. 3. The first layer and the third layer include an expandable material, and the second layer includes a scaffold layer.

The stabilizing cylinder 1004 is configured to be placed in an aneurysm sac 215, as shown in FIG. 2. The stabilizing cylinder may be implemented through one or other suitable methods to treat the abdominal aortic aneurysm and to avoid endoleaks. By using the stabilizing cylinders, blood entering the aneurysm sac 215 can be stopped, which can save a person from a life-threatening rupture.

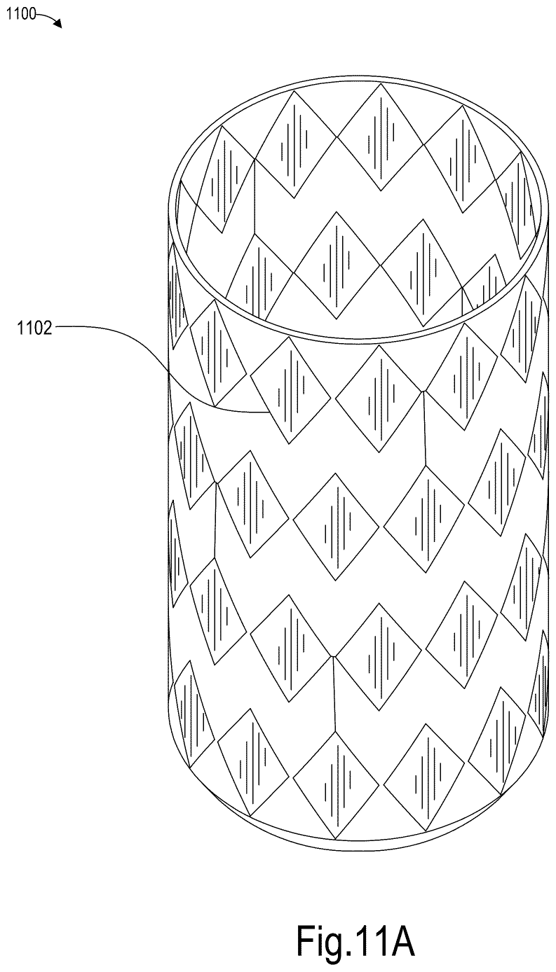

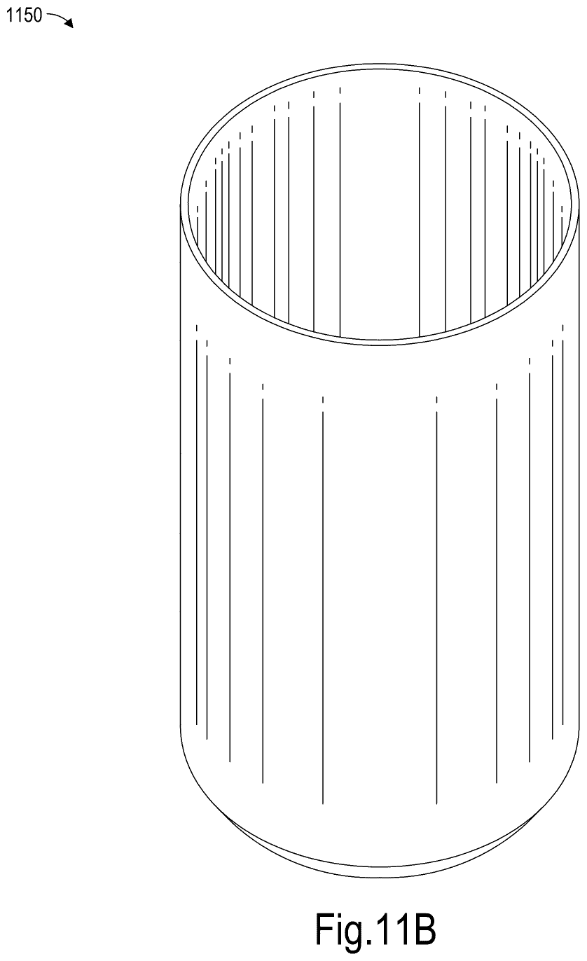

Referring to FIGS. 11A and 11B, FIG. 11A is an image of an embodiment of the stabilizing cylinder 1100 when the axis of the stabilizing cylinder is placed along a vertical axis. FIG. 11B is an image of an embodiment of the stabilizing cylinder 1150 without a scaffold. In an example embodiment, the stabilizing cylinder 1100 may include a first layer of expandable material, a second layer of expandable material layered over the first layer of expandable material, and a scaffold layer 1102 (which appears as a pattern of lines in FIG. 11A) in between the first layer of expandable material and the second layer of expandable material. All three layers are arranged in a cylinder shape.

The stabilizing cylinder 1100 is in an expanded state after being deployed to fill a space within an aneurysmal sac. The three layers are arranged to provide a space within the stabilizing cylinder for a flow of blood. The scaffold layer may be constructed of a material configured to self-expand subsequent to deployment in a blood vessel. In an example embodiment, the scaffold layer comprises nitinol.

Further, all three layers are also arranged in such a manner to position a stent graft within the stabilizing cylinder 1100, which may allow a flow of blood through the aneurysm. The first layer of expandable material and the second layer of expandable material may extend beyond the scaffold layer in a direction opposing the flow of blood in the aneurysm.

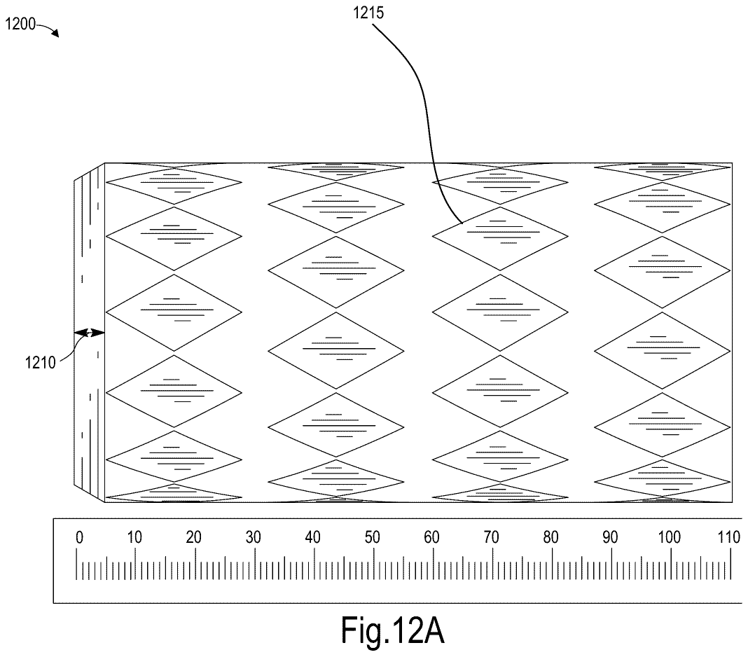

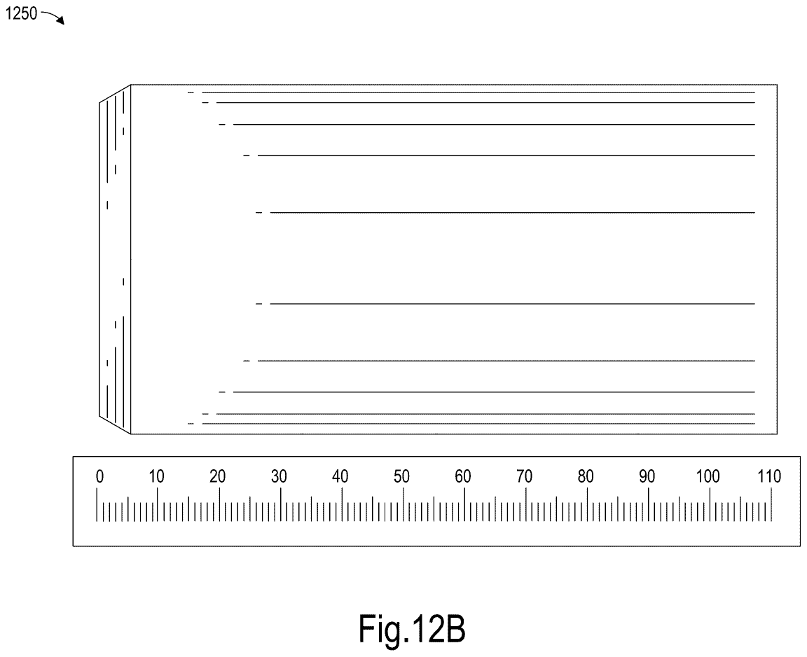

Referring to FIGS. 12A and 12B, FIGS. 12A and 12B are images of embodiments of stabilizing cylinders from a side view with a mm ruler for approximate scale. The stabilizing cylinder 1200 may include a first layer of expandable material, a second layer of expandable material layered over the first layer of expandable material, and a scaffold layer (which appears as a pattern of lines in FIG. 12A) in between the first layer of expandable material and the second layer of expandable material. In example embodiments, the scaffold layer 1215 includes a metal frame. The metal frame may include various materials, such as nitinol and stainless steel. The embodiment of the stabilizing cylinder 1250 shown in FIG. 12B does not include the scaffold layer 1215.

All three layers are arranged in a cylinder shape. In one exemplary embodiment, the first and second layers of expandable material extend beyond the scaffold layer 1215 for a length 1210 at one end of the stabilizing cylinder 1200. The length 1210 vcan be varied based on one or more factors. In an example embodiment, the length 1210 is between about 10 mm and about 20 mm. In an example embodiment, the length 1210 is between about 20 mm and about 30 mm. In another exemplary embodiment, the first and second layers of expandable material extend beyond the scaffold layer 1215 at both ends of the stabilizing cylinder 1200. The length can be varied based on one or more factors.

The stabilizing cylinder 1200 may compressed into a compressed stated during delivery and may expand into an expanded state after being deployed to fill a space within an aneurysmal sac. All three layers are arranged in such a manner to provide a space within the stabilizing cylinder for one or more structures. The scaffold layer is made of a material such that the scaffold layer is configured to self-expand subsequent to deployment in a blood vessel. In one embodiment, the scaffold layer is made of nitinol.

The three layers may be arranged to position a stent graft within the stabilizing cylinder 1200 to facilitate a flow of blood through the aneurysm. The scaffold layer 1215 is positioned such that the first layer of expandable material and the second layer of expandable material extend beyond the scaffold layer in a direction opposing the flow of blood in the aneurysm.

Many variations may be made to the embodiments described herein. All variations, including combinations of embodiments, are intended to be included within the scope of this disclosure. The description of the embodiments herein can be practiced in many ways. Any terminology used herein should not be construed as restricting the features or aspects of the disclosed subject matter. The scope should instead be construed in accordance with the appended claims.

1. A system for treating an aneurysm, the system comprising:
a set of one or more stabilizing cylinders, each stabilizing cylinder comprising:
a first sheet of expandable material;
a second sheet of expandable material layered over the first sheet of expandable material;
a scaffold layer in between the first sheet of expandable material and the second sheet of expandable material; and
combined layers comprising the first sheet of expandable material, the second sheet of expandable material, and the scaffold layer are arranged in a cylinder shape;
each stabilizing cylinder is configured to expand after being deployed to fill a space within an aneurysmal sac; and
each stabilizing cylinder is further configured to provide a space within the stabilizing cylinder for one or more structures.

2. The system of claim 1, wherein the set of one or more stabilizing cylinders comprises two or more stabilizing cylinders.

3. The system of claim 2, wherein lengths of the stabilizing cylinders, as measured along a flow of blood through the aneurysm, are tapered such that the lengths decrease from an inside stabilizing cylinder to an outside stabilizing cylinder.

4. The system of claim 3, wherein tapering of the stabilizing cylinders is configured to fit the stabilizing cylinders within a space in the aneurysm.

5. The system of claim 1, wherein the scaffold layer comprises a material that is configured to self-expand subsequent to deployment in a blood vessel.

6. The system of claim 5, wherein the scaffold layer comprises nitinol.

7. The system of claim 1, further comprising a stent graft that is configured to be inserted inside the set of one or more stabilizing cylinders, the stent graft further configured to facilitate a flow of blood through the aneurysm.

8. The system of claim 1, wherein the scaffold layer of each stabilizing cylinder is positioned such that the first sheet of expandable material and the second sheet of expandable material extend beyond the scaffold layer in a direction opposing a flow of blood in the aneurysm when the stabilizing cylinder is deployed.

9. The system of claim 1, wherein at least one stabilizing cylinder of the set of one or more stabilizing cylinders comprises radiopaque markers.

10. A stabilizing cylinder for treating an aneurysm, the stabilizing cylinder comprising:
a first sheet of expandable material;
a second sheet of expandable material layered over the first sheet of expandable material;
a scaffold layer in between the first sheet of expandable material and the second sheet of expandable material;
combined layers, comprising the first sheet of expandable material, the second sheet of expandable material, and the scaffold layer, are arranged in a cylinder shape;
the combined layers are configured to expand after being deployed to fill a space within an aneurysmal sac; and
the combined layers are further configured to provide a space within the stabilizing cylinder for one or more structures.

11. The stabilizing cylinder of claim 10, wherein the stabilizing cylinder is configured to be layered within the aneurysm with one or more additional stabilizing cylinders.

12. The stabilizing cylinder of claim 11, wherein lengths of the stabilizing cylinder and one or more additional stabilizing cylinders, as measured along a flow of blood through the aneurysm, are tapered such that the lengths decrease from an inside stabilizing cylinder to an outside stabilizing cylinder.

13. The stabilizing cylinder of claim 12, wherein tapering of the stabilizing cylinders is configured to fit the stabilizing cylinders within a space in the aneurysm.

14. The stabilizing cylinder of claim 10, wherein the scaffold layer comprises a material that is configured to self-expand subsequent to deployment in a blood vessel.

15. The stabilizing cylinder of claim 14, wherein the scaffold layer comprises nitinol.

16. The stabilizing cylinder of claim 10, wherein the stabilizing cylinder is configured to receive a stent graft positioned within the stabilizing cylinder to facilitate a flow of blood through the aneurysm.

17. The stabilizing cylinder of claim 10, wherein the scaffold layer is positioned such that the first sheet of expandable material and the second sheet of expandable material extend beyond the scaffold layer in a direction opposing a flow of blood in the aneurysm when the stabilizing cylinder is deployed.

18. The stabilizing cylinder of claim 10, further comprising radiopaque markers.

19. The stabilizing cylinder of claim 10, wherein the first sheet of expandable material and the second sheet of expandable material comprise a foam material.

20. A system for treating an aneurysm, the system comprising:
a set of one or more stabilizing cylinders, each stabilizing cylinder comprising:
a first sheet of expandable material;
a second sheet of expandable material layered over the first sheet of expandable material;
a scaffold layer in between the first sheet of expandable material and the second sheet of expandable material; and
combined layers, comprising the first sheet of expandable material, the second sheet of expandable material, and the scaffold layer are arranged in a cylinder shape;
each stabilizing cylinder is configured to expand after being deployed to fill a space within an aneurysmal sac;
each stabilizing cylinder is further configured to provide a space within the stabilizing cylinder for one or more structures;
wherein the set of one or more stabilizing cylinders comprises two or more stabilizing cylinders;
wherein lengths of the stabilizing cylinders, as measured along a flow of blood through the aneurysm, are tapered such that the lengths decrease from an inside stabilizing cylinder to an outside stabilizing cylinder;
wherein tapering of the stabilizing cylinders is configured to fit the stabilizing cylinders within a space in the aneurysm;
wherein the scaffold layer comprises a material that is configured to self-expand after deployment in a blood vessel; and
wherein the scaffold layer comprises nitinol.
